(19) United States
(12) Patent Application Publication
(10) Pub. No.: US 2003/0050653 A1
(43) Pub. Date: Mar. 13, 2003

(54) Device for holding a sleeve and a method for manufacturing such a device

(76) Inventor: Fritz Berger, Thun (CH)

Correspondence Address: SHOEMAKER AND MATTARE, LTD., Suite 1203 Crystal Plaza Bldg. 1, 2001 Jefferson Davis Highway, P.O. Box 2286, Arlington, VA, 22202-0286, US

(21) Appl. No.: 09/801,931

(22) Filed: Mar. 9, 2001

(51) Int. Cl. A61B 17/08

(52) U.S. Cl. 606/151

(57) ABSTRACT

A device for holding a sleeve penetrating the surface of a human or animal body on the surface consists essentially of two sections (4a, 4bThe two sections are connected to one another via a resilient connection (5) and in each case provided wit longitudinal opening (b) defining a longitudinal direction (La, Lb). In a clamping position (K) the longitudinal dire w (La, Lb) of the longitudinal openings (3a, 3b) of the sections (4a, 4h) run at an angle <<x) to one another. In a displacement position (V) the longitudinal directions (la, Lb) run roughly parallel to one another. A sleeve (2) inserted into the longitudinal opening (3) may be displaced in the longitudinal opening (3) when the holding device is located in the displacement position (V). If the holding device is located in the clamping position (K) the sleeve is non-displaceably clamped.

The invention relates to a device for holding a sleeve penetrating the surface of a human or animal body on this surface and to a method for manufacturing such a device with the features of the independent patent claims.

With the carrying out of surgical operations often for supporting the surgical operation or for diagnostic purposes endoscopes are applied. With this the surface of the human or animal body is penetrated by a sleeve. The sleeve serves for indroducing probes, surgical auxiliary instruments and a gas (insufflation) into a body cavity. Typically the sleeve penetrates the abdominal wall in order to introduce auxiliary means or gases into the abdominal cavity.

There are known devices which serve for fastening the sleeve penetrating the surface on the skin. Such known devices comprise a passage opening for the sleeve in which the sleeve is displaceably inserted. These holding devices serve for holding the sleeve in a certain position and also for closing the passage opening in the surface of the body.

Usually the holding device by way of a thread attached in the abdominal wall is fastened on the surface of the body. So that the sleeve may be rigidly held in a certain position with respect to the abdominal wall, the known holding devices are provided with a clamping mechanism. The clamping mechanism permits the sleeve to be displaced in the passage opening of the holding device and to be fastened in a desired position. The clamping mechanism with known devices consists of several components connected to one another. On account of the connections (which often are designed as articulated connections) the cleaning and sterilisation of such holding devices is not possible or only with difficulty.

On account of the multi-part design also the manufacture of the known holding devices is complicated and these are therefore expensive.

It is an object of the present invention to avoid the disadvantages of that which is known, in particular to provide a device for holding a sleeve which may be used several times, which is manufacturable in a simple manner and which is suitable for cleaning and sterilisation. A further object of the present invention lies in providing a method for manufacturing such a holding device which may be carried out simply and in an economical manner.

According to the invention these objects are achieved with a device and with a method with the features of the characterising part of the independent patent claims.

The device comprises at least two sections which are connected to one another with a resilient connection. In each of the sections there is incorporated a part of the longitudinal opening running in a longitudinal direction. The resilient connection is at the same time selected in a manner such that the sections in a clamping position clamp on an inserted sleeve and that the sections may be brought against the spring force into a displacement position in which the sleeve is displaceable through the longitudinal opening.

In a preferred embodiment example in a clamping position the mutual position of the sections is selected in a manner such that the longitudinal directions of the longitudinal openings of the two sections run at an angle to one another. In a displacement position the longitudinal directions of the longitudinal openings of the two sections run roughly parallel to one another.

Because in the displacement position the longitudinal directions of the two longitudinal openings run roughly parallel to one another, a sleeve inserted into the longitudinal openings may be simply displaced.

Because the longitudinal directions in the clamping position are at an angle to one another, a sleeve inserted into the longitudinal openings is clamped. On account of this clamping effect the sleeve in the holding device is no longer displaceable. The longitudinal openings in the sections are preferably designed cylindrically since sleeves for endoscopy are usually likewise designed cylindrically.

If the outer diameter of the sleeve is smaller than the free inner diameter of the longitudinal openings the sleeve in the holding device is then still displaceable when the longitudinal directions of the two longitudinal openings are at a small angle to one another. As soon as this angle however becomes too large al so with such a dimensioning there results a clamping effect.

It is however preferred that the free inner diameter of the longitudinal openings is the same size as the outer diameter of the sleeve.

In a particularly preferred embodiment example in the clamping position the resilient connection is essentially relaxed and in the displacement position the resilient connection is tensioned.

In particular in the displacement position the two sections are advantageously pressed against one another. It is however also conceivable that for achieving the displacement position the i sections are pulled apart against the resilient connection.

The resilient connection is with this in the clamping position not completely relaxed. In this embodiment example it is important that the device with the absence of an external force effect automatically assumes the clamping position. The advantage of this embodiment example lies in the fact that for displacing the sleeve the two sections are tensioned against one another, for example must be pressed against one another and that without doing anything automatically always the clamping position is assumed.

Advantageously the device is designed as one piece, for example of a sterilisable plastic material. This is with regard to a simple manufacture and a simple sterilisability particularly favourable. It would however also be conceivable to provide a holding device which consists of two sections which are connected to one another in an articulated manner and which with an inserted spring may be tensioned against one another. In order to ensure sterilisability it is sufficient to design the individual components easily releasable from one another.

It is also conceivable to provide sections which are displaceable laterally to one another and with which in the displacement position the openings are in alignment and in the clamping position are offset.

It is furthermore also conceivable to form the holding device of metal, for example stainless steel. With a holding piece designed as one piece the resilient connection may be particularly simply formed by a bending joint formed as one piece with the two sections. The sections may be separated from one another by a gap running roughly perpendicular to the longitudinal direction.

For simplifying the handling of the holding device it is furthermore advantageous to provide the sections with projecting grip tabs by way of which the sections may be pressed together against one another or where appropriate also pulled apart.

The device may furthermore in the known manner be provided with a roughly conical sealing part which serves for closing the passage opening in the surface of the body. Furthermore the device may comprise holding projections which serve for fastening a thread sewn into the skin. The holding projections may have a substantially disk-like shape and a gap for fastening the thread may be formed between a surface of the device and a peripheral portion of the disk-like holding projections. Threads thereby may simply be clamped between the surface of the device and the disk-like holding projection in this gap.

According to a preferred embodiement of the invention, the thickness of the gap is increasing radially outwardly. Threads with different thichnesses may be easily attached on such a projection. Due to the reseiliency of the holding projections, the disk-like holding projection may be bent slightly outwardly when the threads are introduced into the gap and clamps said thread in the gap due to its resiliency.

In place of sleeves in the actual sense, of course with the mounting according to the invention also other medical apparatus such as optical conductors, tubings or the like may be held which penetrate the surface of a body.

The method for manufacturing the previously described device lies essentially in that firstly there is prepared a blank and then subsequently into the blank there are incorportated the longitudinal openings.

According to the invention a blank is prepared which comprises two sections which are resiliently connected to one another. Before incorporating the longitudinal openings the sections are brought into a displacement position against the resilient connection, typically pressed together. Whilst the sections are located in the displacement position a longitudinal opening penetrating the two sections, in particular a bore, is incorporated. With this manufacturing method it is ensured that in the displacement position the longitudinal directions of the longitudinal openings in the two sections run parallel to one another. The two sections may for example be separated from one another in that in the blank a gap is incorporated, for example milled in. Preferably the two sections after incorporating the through-running longitudinal opening (e.g. after removal of a drill) are held further in the displacement position. Subsequently a sleeve is inserted into the longitudinal opening. This is particularly simply possible since the holding device is still located in the displacement position. As soon as the sections are no longer held in the displacement position on account of the resilient connection they return back into a position defining the clamping position and fix the previously inserted sleeve.

The invention is hereinafter described in more detail by way of the drawings. There are shown in:

FIG. 1 a schematic representation of a sleeve and of a holding device in the lateral view,

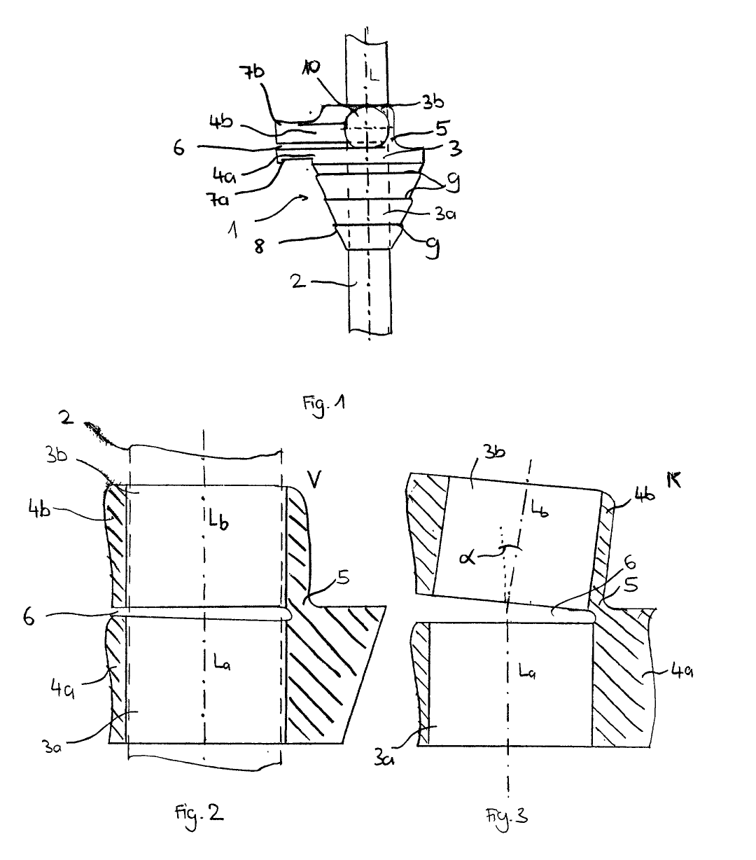

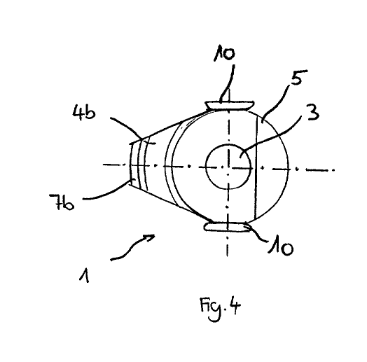

FIG. 2 a cross section through a holding device in a displacement position,

FIG. 3 a cross section through the holding device in the clamping position,

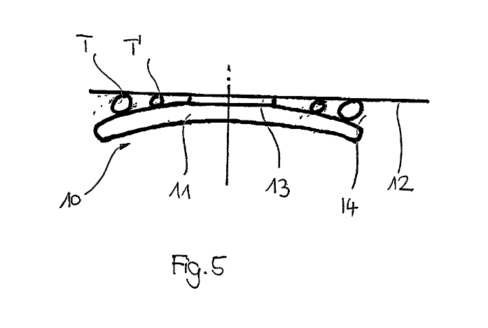

FIG. 4 a plan view of the holding device according to the invention, and

FIG. 5 an enlarged view of a holding projection.

FIG. 1 shows a holding device 1 which is provided with a through-going longitudinal bore 3. Into the through-going longitudinal bore 3 there is inserted a sleeve 2. The sleeve 2 consist for example of stainless steel.

The device 1 consists essentially of a lower section 4a and of an upper section 4b. The sections 4a, 4b are via a bending joint resiliently connected to one another.

The lower section 4a is provided with a first longitudinal opening 3a and the upper section 4b with a second longitudinal opening 3b. The longitudinal openings 3a, 3b together form the through-going longitudinal bore 3 through the holding device 1. The two sections 4a, 4b are separated from one another by a gap 6 running roughly perpendicular to the longitudinal direction L of the longitudinal bore 3. On account of the gap 6 the two sections 4a, 4b may be pressed together against the spring force mustered by the bending joint 5. For the pressing together of the sections 4a, 4b these are provided with grip tabs 7a, 7b which project laterally outwards.

The holding device 1 is furthermore provided with a roughly conical sealing surface 8. The sealing surface 8 may furthermore be provided with ribs 9. The sealing surface 8 serves for closing the penetration opening in the surface of the body through which the sleeve 2 penetrates into a body cavity.

Furthermore on the holding device 1 in the upper section 3b there are provided holding projections 10 around which a thread sewn into the skin may be wound for holding the holding device on the surface of the body (see also Figures). On account of the resilient connection the two sections 4a, 4b tend to assume a relaxed position. This relaxed position defines a clamping position K (see FIG. 3), in which the longitudinal directions 3a, 3b or the sections 4a, 4b are at an angle to one another. A sleeve 2 introduced into the longitudinal opening 3 on account of the longitudinal openings 3a, 3b being at an angle to one another is clamped and may no longer be displaced. For pressing together the sections 4a, 4b in the region of the grip tabs 7a, 7b the longitudinal directions La, Lb of the longitudinal openings 3a, 3b are aligned to one another. The longitinal directions La, Lb which are aligned to one another define a displacement position V in which the sleeve 2 may be displaced in the holding device 1.

FIG. 2 shows a cross section through the holding device 1 in the displacement position V.

The two sections 4a, 4b are pressed together against the spring force of the bending joint 5. The longitudinal direction La of the longitudinal opening 3a of the lower section 4a runs in the displacement position V parallel to the longitudinal direction Lb of the longitudinal opening 3b of the upper section 4b. The sleeve 2 may thus be simply displaced in the holding device 1. In FIG. 3 there is shown a cross section through the holding device in a clamping position K. In the clamping position the resilient connection between the lower section 4a and the upper section 4b created by the bending joint 5 is somewhat relaxed. The longitudinal axis La of the longitudinal opening 3a of the lower section 4a runs at an angle α to the longitudinal direction Lb of the longitudinal opening 3b of the upper section 4b.

In FIG. 3 the angle a for representational reasons is shown greatly exaggerated. The angle α in reality is about 3 to 6 degrees.

On account of the longitudinal openings 3a, 3b running in different directions a sleeve inserted into the through-going longitudinal opening 3 is clamped. On account of the resilient connection between the sections 4a, 4b these remain in the clamping position without external force effect. The sleeve may only be displaced when the two sections 4a, 4b are brought against the spring force into the displacement position shown in FIG. 2.

In FIG. 4 there is shown a plan view of the holding device 1.

Laterally on the holding device 1 there are provided holding projections 10 around which a thread sewn into the skin may be wound for fastening the holding device.

The longitudinal opening 3 is formed as a bore which serves for receiving a usual sleeve. The bore has thus a diameter of about 10-15 mm. Laterally projecting are grip surfaces 7b and 7a (in Figure hidden) which serve for pressing together the sections 4a, 4b against one another.

FIG. 5 shows an enlarged view of the holding projection 10 shown in FIG. 4. The holding projections comprise a disk-like portion 11 which is connected to the surface 12 of the device 1 with a cylindrical connector 13. A gap 14 is formed between the disk-like portion 11 and the surface 12 of the device 1. Th reads T, T′ of different sizes may be wound around the cylindrical connector 13 and may be clamped in the gap 14 which has a thickness increasing radially outwardly. When the threads T or T′ are wound around the holding device, the disk-like portion 11 is flexed slightly outwardly due to the resiliency of the material. Threads of different thicknesses thereby may be safely fixed within the gap 14. The holding device 1 is typically manufactured from a blank of a sterilisable plastic material, such as ertacetal. Into the blank firstly a gap 6 (see FIGS. 1 to 3) is milled which extends essentially transversely through the holding device so that there are formed two sections 4a, 4b formed by a bending joint 5. Before incorporating a through-going longitudinal bore 3 the sections 4a, 4b are pressed together against the spring force of the bending joint 5 into the displacement position V. The through-going longitudinal opening 3 is incorporated into the displacement position V, typically drilled. After removing the drill the holding device is left in the displacement position and the sleeve 2 is inserted. After insertion the sections 4a, 4b may be let go of so that they are placed back as far as possible into the original position on account of the resilient connection. A complete placing back into the original position on account of the inserted sleeve 2 may not be possible. The position assumed after letting go of the sections 4a, 4b defines a clamping position in which the sleeve is not displaceable in the holding device.

1. A device for holding a sleeve penetrating a surface, such as the abdominal wall, of a human or animal body, on the surface
said device comprising a body with a through-going, longitudinal opening formed therein in which the sleeve is displaceably insertable,
the device having at least two sections
each of said sections including a part of said longitudinal opening
whereby said sections are resiliently connected to one another
whereby in a clamping position said sections clamp on the inserted sleeve and in a displacement position said sleeve is displaceable in the longitudinal direction and
whereby said sections may be brought into said displaceable position against a spring force.

2. A device according to claim 1, whereby said sections in each case have a longitudinal opening and wherein in said clamping position the mutual position of said sections is selected in a manner such the longitudinal openings of the sections have longitudinal directions that are at an angle to one another so that a sleeve inserted into the longitudinal opening is clamped and
wherein in said displacement position said longitudinal directions run roughly parallel to one another so that a sleeve inserted into the longitudinal opening is displaceable.

3. A device according to claim 1, wherein the device is designed as one piece.

4. A device according to claim 3, wherein the device is made of a sterilisable plastic material.

5. A device according to claim 3, wherein said resilient connection is formed as a bending joint formed with said sections, wherein said sections are separated from one another by a gap running roughly perpendicular to the longitudinal direction of the longitudinal openings.

6. A device according claim 1, wherein said sections are each provided with a projecting grip tab by way of which the said sections can be pressed together against one another.

7. A device according claim 1, wherein the device has a roughly conical sealing part.

8. A device according to claim 1, wherein the device is provided with at least one holding projection for fastening a thread.

9. A device according to claim 8, wherein said at least one holding projection has a substantially disk-like shape, whereby a gap for fastening said thread is formed between a surface of said device and a peripheral portion of said

10. A device according to claim 9, wherein said gap has a thickness increasing radially outwardly.

11. A method for manufacturing a device for holding a sleeve penetrating a surface comprising the steps of
preparing a blank with two sections resiliently connected to one another,
tensioning, the sections against a resilient connection into a displacement position
incorporating a longitudinal opening penetrating both sections.

12. A method according to claim 11, comprising the further step of separating said section from one another by incorporating, a gap in the blank.

13. A method according to claim 11, comprising the steps of holding said sections after incorporating the longitudinal opening in said displacement position and subsequently inserting a sleeve into said longitudinal opening.
